(19) United States
(12) Patent Application Publication
(10) Pub. No.: US 2006/0160841 A1
(43) Pub. Date: Jul. 20, 2006

(54) Crystallization via high-shear transformation

(76) Inventors: Chenkou Wei, Princeton Junction, NJ (US); Bing-Shiou Yang, Dayton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/235,327

(22) Filed: Sep. 26, 2005

Related U.S. Application Data
(60) Provisional application No. 60/645,056, filed on Jan. 19, 2005.

(51) Int. Cl. C07D 471/02; A61K 31/4745

(52) U.S. Cl. 514/303; 546/118

(57) ABSTRACT

The invention relates to a process or apparatus for transforming a first polymorph of a chemical material into a second polymorph of the same chemical material, utilizing an apparatus comprising a vessel connected to a re-circulation system, the process comprising the steps of: suspending said first polymorph in a solution to form a slurry in the vessel, re-circulating the slurry and removing the slurry from the vessel.

### CROSS REFERENCE TO RELATED APPLICATIONS

This application claims a benefit of priority from U.S. Provisional Application No. 60/645,056 filed Jan. 19, 2005, the entire disclosure of which is herein incorporated by reference

### FIELD OF THE INVENTION

The present invention relates to a process for preparing small crystals of organic pharmaceutical compounds and more particularly to a crystallization process that utilizes high-shear assisted polymorph transformation and to the apparatus for practicing such a process.

### BACKGROUND OF THE INVENTION

It is well known in the pharmaceutical industry that the bioavailability of a sparingly soluble organic compound is often enhanced when the compound is very pure and the molecules of the compound have a small, uniform particle size, high surface area, and short dissolution time. Purification can be accomplished by crystallization of the compound from solution. However, when crystallization takes place directly in a high supersaturation environment, the resulting material is often unsatisfactory due to low purity, high friability, and lack of stability because the crystal structure formation is inadequate. Further, oils commonly produced during processing of supersaturated material may solidify without sufficient structure.

It is possible to slow down the crystallization process to obtain a higher purity, more stable product. However, slowing the process decreases crystallizer productivity and produces particles which are too large, having low surface area. Such particles require high intensity milling to create a useable product.

To overcome those problems, and provide crystalline particles of high surface area, high chemical purity, and high stability, without the need for post-crystallization milling, a crystallization process, known as the “impinging fluid jet” process, has been developed.

One well known version of the “impinging fluid jet” process is disclosed in detail in U.S. Pat. No. 5,314,506 entitled “Crystallization Method To Improve Crystal Structure And Size” issued May 24, 1994, to Midler et al., owned by Merck & Co., Inc. of Rahway, N.J. The reader is referred to that patent for background information and the details of the process.

Basically, the impinging fluid jet process utilizes a supersaturated solution of the compound to be crystallized in solvent and an appropriate “anti-solvent” solution. Diametrically opposed high velocity jet streams of these solutions are formed by nozzles and micro mixed in a jet chamber. The mixed solutions are then transferred into a vessel where they are stirred to produce the end product. The product, such as a neutral molecule or a salt, is crystallized out by mixing the solutions which reduces the solubility of the compound in the solvent mixture.

The impinging fluid jet stream process has also been used for conducting reactive crystallization wherein a chemical reaction and controlled crystallization take place simultaneously. Patent Application Publication No.: U.S. 2002/0016498 A1 of Feb. 7, 2002, entitled “Reactive Crystallization Method to Improve Particle Size” in the name of Am Ende et al., owned by Pfizer Inc., provides further information in this regard.

Reactive crystallization involves two reactive intermediates. Fluid streams of solutions of the reactive intermediates are impinged in a chamber under appropriate reactive conditions. For example, a first solution containing one reagent (for example, an acid) in a solvent is reacted with a second solution containing another reagent (for example, a base) in a solvent are reacted to form a product, such as a salt. The product is not soluble in the solvent mixture and thus it rapidly crystallizes out. In the pharmaceutical industry, the drug substance is often in a salt form, so reactive crystallization is commonly used.

Recently, Wei et al. disclosed a crystallization system using homogenization in WO 03/095059 and another crystallization system utilizing atomization in WO 03/092852. The entire disclosures of each of the aforementioned patents or patent application publications are incorporated herein by reference.

There remains a need to develop a robust crystallization process that can produce small and uniform crystals with high purity, high stability, and high surface area, and without the necessity of post-crystallization milling.

### SUMMARY OF THE INVENTION

The present invention relates a process for transforming a first polymorph of a chemical material into a second polymorph of the same chemical material, utilizing an apparatus comprising a vessel connected to a re-circulation system, the process comprising the steps of: suspending the first polymorph in a solution to form a slurry in the vessel, re-circulating the slurry, and removing the slurry from the vessel.

The present invention relates to apparatus for transforming a first polymorph of a chemical material into a second polymorph of the same chemical material.

### BRIEF DESCRIPTION OF THE DRAWINGS

To these and such other objects which may hereinafter appear, the present invention relates to a process transforming a polymorph (including solvate) of a chemical material into a second polymorph (including solvate) of the same chemical material, using a vessel connected to a recirculation system. The recirculation can be conducted through a homogenization apparatus in which a high-shear force is applied, as described in detail in the following specification and recited in the annexed claims, taken together with the accompanying drawings, wherein like numerals refer to like parts and in which:

FIG. 1 is a schematic drawing of an apparatus utilized to perform the process of the present invention;

FIG. 2 is a cross-sectional view of homogenization chamber G with rotor H and stator I. J and K are the inlet and outlet of the slurry, respectively;

FIG. 3 is a cross-sectional view taken along line 1-1 of FIG. 2;

FIG. 4 is a schematic drawing of an apparatus that can be utilized to perform the process of the present invention; and

FIG. 5 is a schematic drawing of an apparatus that can be utilized to perform the process of the present invention.

### DETAILED DESCRIPTION OF THE INVENTION

In accordance with one aspect of the present invention, the process utilizes an apparatus comprising a vessel connected to a re-circulation system. The process is useful for transforming a first polymorph (including solvate) of a chemical material into a second polymorph (including solvate) of the same chemical material. The second polymorph can be thermodynamically more stable or thermodynamically less stable than the first polymorph. In addition, the first polymorph can be in a solvate form, including hydrate form and the second polymorph can be in an anhydrous form. Further, the present invention can be especially effective for transforming a first polymorph which consists of large crystals into a second polymorph which consists of small crystals. Generally, large crystals have a particle size D[90] greater than about 100 μm, and small crystals have a particle size D[90] less than about 30 μm. In addition, large crystals can have a particle size D[90] greater than about 60 μm, and small crystals can have a particle size D[90] less than about 50 μm.

The recirculation can be conducted through a homogenization apparatus in which a high-shear force is applied. The homogenization apparatus comprises a stator and a rotatable rotor, and the high-shear mixing force is applied by rotating the rotor at a speed of more than 250 rpm. The rotor can also be rotated as speeds of more than 500 rpm and more than 1,000 rpm.

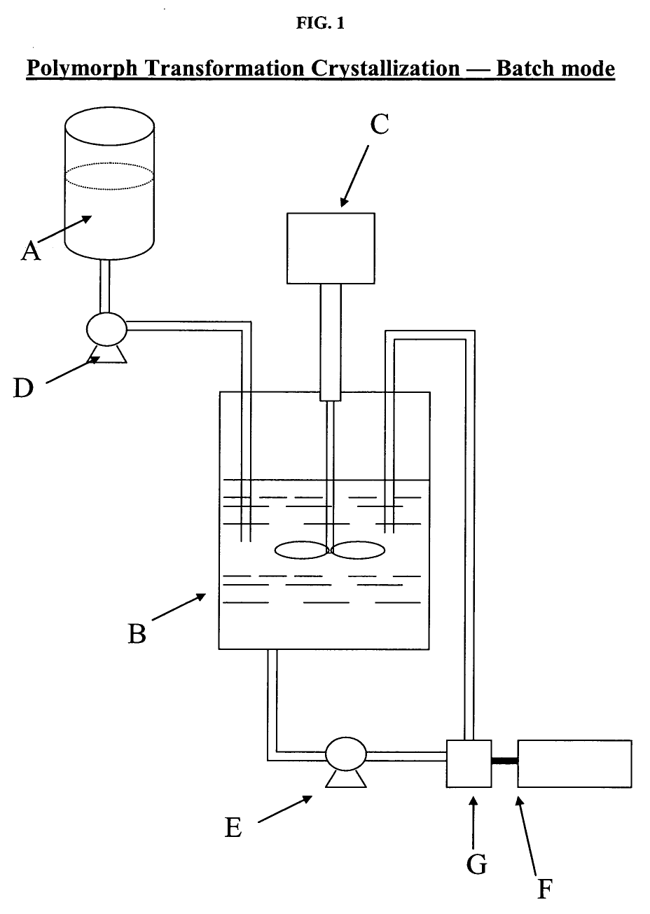

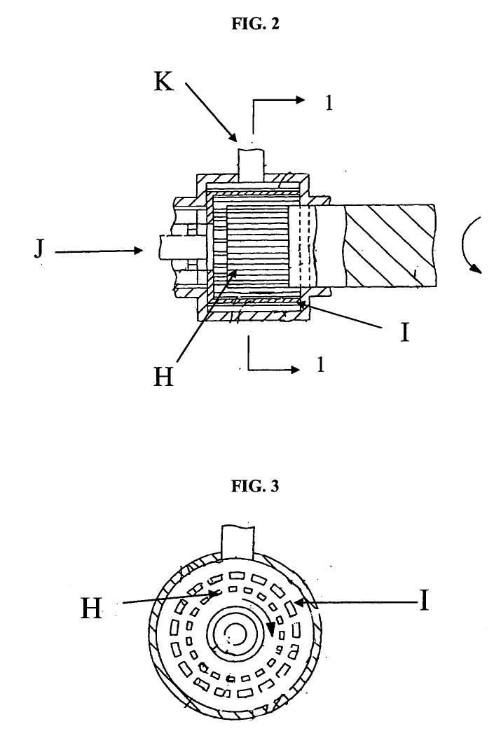

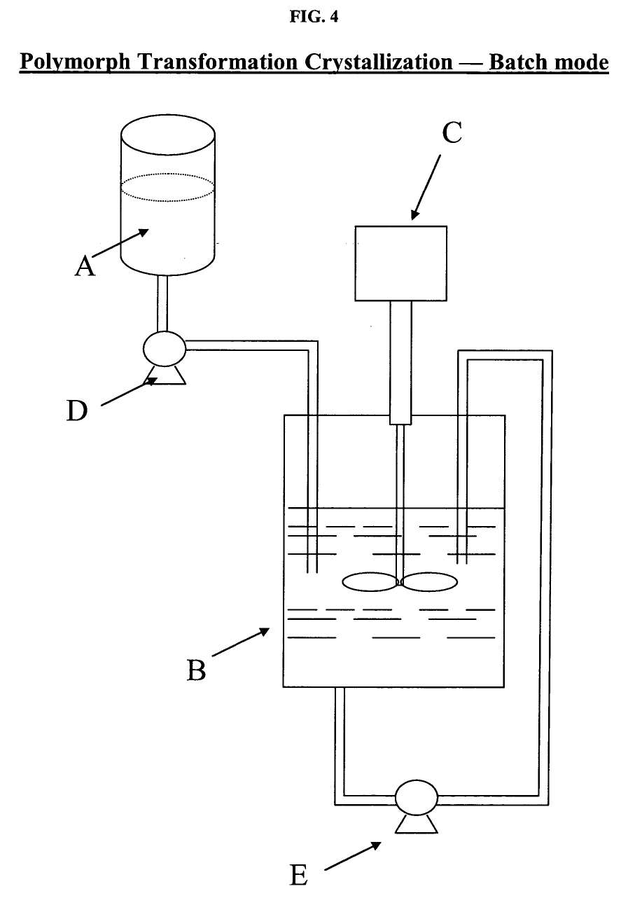

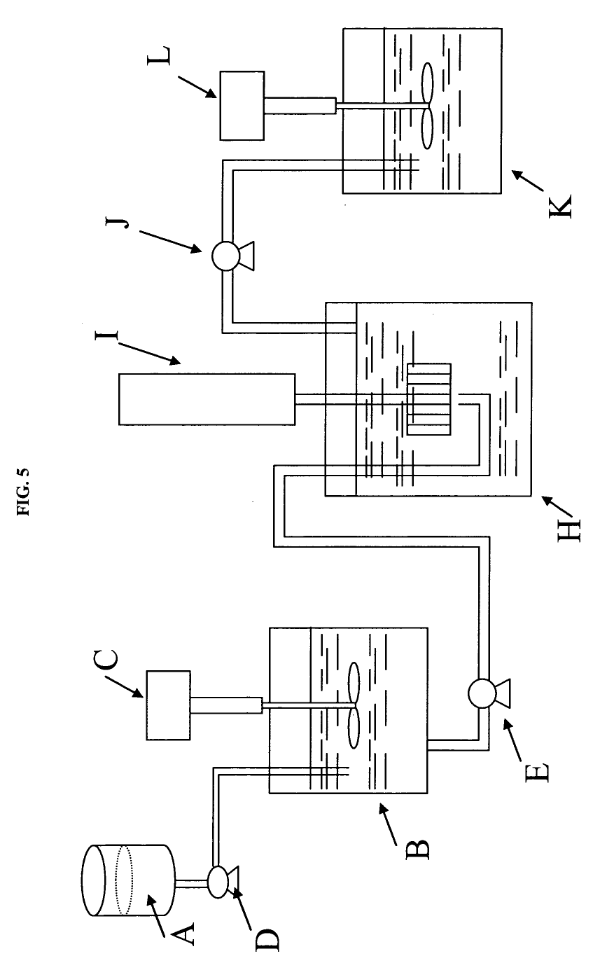

Re-circulating the slurry comprises regulating the flow of slurry through the outlet and the inlet of the vessel, as illustrated in FIGS. 1 and 5. The energy for the re-circulation can be provided by a pump. Conventional flow regulation mechanisms such as metering pumps, valves, and the like may be used for this purpose. The process can also be conduced in a continuous mode, as illustrated in FIGS. 1 and 4.

Suspending the large crystals in a solution to form a slurry in the vessel comprises adjusting the temperature of the solution in the vessel. This may be achieved by any conventional temperature adjusting equipment, such as a heater or a cooling bath associated with the vessel.

In accordance with another aspect of the present invention, the process is useful for producing small crystals of a chemical material through polymorph transformation, utilizing the apparatus as described above and as illustrated in FIGS. 1, 4, and 5. The process comprises the steps of: (a) mixing a first solution into a second solution in the vessel (e.g., vessel A) to form a slurry (i.e., first polymorph), wherein the first solution comprises the material to be dissolved in a solvent (e.g., propylene glycol (PG)) and the second solution comprises an anti-solvent (e.g., water); (b) re-circulating the slurry, and (c) removing the slurry from the vessel after the second polymorph has been formed.

The first solution may be a supersaturated solution comprising the chemical material to be crystallized, such as a neutral molecule or a salt, dissolved in a solvent. This material-containing solution is mixed with a second solution, which is an anti-solvent solution. The anti-solvent refers to any solvent in which the chemical material has a poor solubility. It may be a mixture of anti-solvents and solvents. For example, the anti-solvent may comprise water and PG. Mixing the solutions reduces the solubility of the material in the solvent mixture, causing it to crystallize out. Optionally, the second solution may contain a limited amount of the chemical material.

The process may also include the step of introducing seed crystals into the vessel to facilitate crystallization. The seed crystals may be placed into the vessel prior to the introduction of the solutions or seed crystals may be added to one of the solutions prior to its introduction into the vessel. These seed crystals must be insoluble in the individual solvents and in the solvent mixture.

As used herein, the terms “first” and “second” are not intended to denote order or to limit the invention to a particular sequence of the combination of the constituents. Further, the term “solution” is used generically and should be understood to include dispersions, emulsions, multi-phase systems, and pure solvents, as well as solutions.

Mixing the first solution with the second solution in the vessel comprises regulating the flow of each of the solutions into the vessel. Conventional flow regulation mechanisms such as metering pumps, valves, and the like may be used for this purpose.

The temperature of one or both of the solutions may be adjusted prior to their introduction into the vessel. This may be achieved by any conventional temperature adjusting equipment, such as a heater or a cooling bath associated with the solution source.

In accordance with another aspect of the present invention, the process may also utilize an apparatus comprising a first vessel, a second vessel, and a third vessel as illustrated in FIG. 5. The process comprising the steps of: (a) mixing a first solution into a second solution in the first vessel to form a slurry, wherein the first solution comprises the material to be crystallized dissolved in a solvent and the second solution comprises an anti-solvent; (b) transferring the slurry into the second vessel wherein a high-shear mixing force is applied on the slurry via a homogenizer apparatus; (c) transferring the resulting slurry from the second vessel into the third vessel and agitating, and (d) removing the slurry from the third vessel.

The temperature of the first and/or second solutions may be adjusted prior to their introduction into the first vessel. In addition, the temperature of one or more of the vessels may be adjusted. This may be achieved by any conventional temperature adjusting equipment, such as a heater or a cooling bath associated with the solution source or the vessel.

Mixing the first solution with the second solution in the first vessel comprises regulating the flow of each of the solutions into the vessel. Further, transferring the slurry from the first vessel into the second vessel and from the second vessel into the third vessel comprises regulating the flow of each of the slurry into each vessel. Conventional flow regulation mechanisms such as metering pumps, valves, and the like may be used for this purpose.

In accordance with another aspect of the present invention, an apparatus is provided for transforming a first polymorph (including solvate) of a chemical material into a second polymorph (including solvate) of the same chemical material, comprising a vessel connected to a re-circulation system; a means for suspending the first polymorph in a solution to form a slurry in the vessel; a means for re-circulating the slurry; and optionally a means for removing the slurry from the vessel. Removing can be performed by decanting or other similar procedures. Thus, a means for removing is optional.

The re-circulation system comprises a homogenization apparatus; outlet means for transferring the slurry from the vessel to the homogenization apparatus; and inlet means for receiving the slurry from the homogenization apparatus into the vessel. The homogenization apparatus comprises a stator and a rotatable rotor, and means for applying a high-shear mixing force by rotating the rotor. The high-shear mixing force can be applied by rotating the rotor at a speed of more than 250 rpm. The rotor can also be rotated as speeds of more than 500 rpm and more than 1,000 rpm.

The apparatus may include a means for regulating the flow of slurry through the homogenization apparatus. Conventional flow regulation mechanisms such as metering pumps, valves, and the like may be used for this purpose. The apparatus may also include a means for adjusting the temperature of the slurry in the vessel. This may be achieved by any conventional temperature adjusting equipment, such as a heater or a cooling bath associated with the solution source or the vessel.

In accordance with another aspect of the present invention, an apparatus is provided for producing small crystals of a chemical material through polymorph transformation, comprising a first source of a first solution; a second source of a second solution; a vessel connected to a re-circulation system; a means for mixing the first solution with the second solution in the vessel to form a slurry in the vessel; a means for re-circulating the slurry; and optionally a means for removing the slurry from the vessel. Removing can be performed by decanting or other similar procedures. Thus, a means for removing is optional. The first solution comprises the material to be crystallized dissolved in a solvent and the second solution comprises an anti-solvent. The re-circulation system comprises a homogenization apparatus; an outlet means for transferring the slurry from the vessel to the homogenization apparatus; and an inlet means for receiving the slurry from the homogenization apparatus into the vessel. The homogenization apparatus comprises a stator and a rotatable rotor, and a means for applying a high-shear mixing force by rotating the rotor. Said means for applying a high-shear mixing force is achieved by rotating the rotor at a speed of more than 250 rpm. The rotor can also be rotated as speeds of more than 500 rpm and more than 1,000 rpm.

The apparatus may include a means for regulating the flow of slurry through the homogenization apparatus. Conventional flow regulation mechanisms such as metering pumps, valves, and the like may be used for this purpose. The apparatus may also include a means for adjusting the temperature of the slurry in the vessel. This may be achieved by any conventional temperature adjusting equipment, such as a heater or a cooling bath associated with the solution source or the vessel.

In accordance with another aspect of the present invention, an apparatus is provided for producing small crystals of a chemical material through polymorph transformation, comprising a first source of a first solution; a second source of a second solution; first vessel; a second vessel; a third vessel; a means for mixing the first solution with the second solution in the first vessel to form a slurry (i.e., first polymorph) in the first vessel; a means for transferring the slurry from the first vessel into the second vessel; a means for applying a high-shear mixing force on the slurry in the second vessel via a homogenizer apparatus; a means for transferring the resulting slurry from the second vessel into the third vessel; a means for applying agitation on the slurry in the third vessel; and optionally a means for removing the slurry from the third vessel after the second polymorph has been formed. Removing can be performed by decanting or other similar procedures. Thus, a means for removing is optional.

The first solution comprises the material to be crystallized dissolved in a solvent and the second solution comprises an anti-solvent. The re-circulation system comprises a homogenization apparatus; outlet means for transferring the slurry from the vessel to the homogenization apparatus; and inlet means for receiving the slurry from the homogenization apparatus into the vessel. The homogenization apparatus comprises a stator and a rotatable rotor, and means for applying a high-shear mixing force by rotating the rotor. Said means for applying a high-shear mixing force is achieved by rotating the rotor at a speed of more than 250 rpm. The rotor can also be rotated as speeds of more than 500 rpm and more than 1,000 rpm.

The apparatus may include a means for regulating the flow of slurry through the homogenization apparatus. Conventional flow regulation mechanisms such as metering pumps, valves, and the like may be used for this purpose. The apparatus may also include a means for adjusting the temperature of the slurry in the vessel. This may be achieved by any conventional temperature adjusting equipment, such as a heater or a cooling bath associated with the solution source or the vessel.

### EXAMPLES

### Example 1

As illustrated in FIG. 1, 350 grams of 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxamide are dissolved in about 4900 mL propylene glycol (PG) at about 110° C. in supply vessel A to form a solution. The anti-solvent (i.e., 4200 mL of water and 420 mL of PG) is charged into vessel B. With agitation, provided by mixer C, the solution in supply vessel A is pumped in the submerge mode, by a pump D, to vessel B, while maintaining the batch temperature between 10 to 20° C. At this stage, needle-shaped crystals are formed in vessel B. The crystals have a particle size D[90] greater than about 160 μm and are also in the dihydrate (H2-2) form.

After the charge is over, the slurry (i.e., crystals) in vessel B is re-circulated (approximately one tank volume, i.e., 9520 mL, per minute) through the homogenization chamber of an inline homogenization apparatus, such as Turrax, designated F, by pump E. The homogenization apparatus includes a chamber G with a stator and a rotor. After about 24 hours of recirculation, the needle-shaped crystals are transformed into small, granular crystals which have a particle size D[90] less than about 20 μm and which are in the non-solvate N-1 form. The small crystals are filtered and washed with the anti-solvent (water), and dried under vacuum at about 60° C.

### Example 2

As illustrated in FIG. 4, 250 grams of 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxamide are dissolved in about 3500 mL propylene glycol (PG) at about 110° C. in supply vessel A to form a solution. The anti-solvent (i.e., about 3000 mL of water and about 300 mL of PG) is charged into vessel B. With agitation, provided by mixer C, the solution in supply vessel A is pumped in the submerge mode, by a pump D, to vessel B, while maintaining the batch temperature between 10 to 20° C. At this stage, needle-shaped di-hydrate crystals are formed in vessel B.

After the charge is over, the slurry (i.e., crystals) in vessel B is recirculated (approximately one tank volume, i.e., 6800 mL, per minute) through through the outlet and the inlet of vessel B, by pump E. After about 30 hours of recirculation, the large needle-shaped crystals are transformed into small, granular crystals which have a particle size D[90] less than about 20 μm and which are in the non-solvate N-1 form. The small crystals are filtered and washed with the anti-solvent (water), and dried under vacuum at about 60° C.

### Example 3

As illustrated in FIG. 5, 250 grams of 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxamide are dissolved in about 3500 mL propylene glycol (PG) at about 110° C. in supply vessel A to form a solution. The anti-solvent (i.e., about 3000 mL of water and about 300 mL of PG) is charged into vessel B. With agitation, provided by mixer C, the solution in supply vessel A is pumped in the submerge mode, by a pump D, to vessel B, while maintaining the batch temperature between 10 to 20° C. At this stage, needle-shaped di-hydrate crystals are formed in vessel B.

After small N-1 seed crystals are charged into transient vessel H, the slurry (i.e., crystals) in vessel B is transferred through pump E into the transient vessel H while maintaining the tank temperature at 55-65° C. In transient tank H, the slurry is strongly sheared and re-circulated by an overhead type homogenizer I to ensure fast polymorph transformation and production of small granular N-1 crystals. The slurry was continuously decanted by pump J to receiver vessel K while maintaining the residence time of the slurry in vessel K for 5-10 minutes. The receiver vessel K is agitated by mixer L and the temperature is maintained at 55-65° C. during the transfer. After the transfer is over, the slurry in vessel K is cooled to room temperature. The small crystals are filtered, washed with the anti-solvent (water), and dried under vacuum at about 60° C. to give small, granular crystals which have a particle size D[90] less than about 20 μm and which are in the non-solvate N-1 form.

We claim:

1. A process for transforming a first polymorph of a chemical material into a second polymorph of the same chemical material, utilizing an apparatus comprising: a vessel connected to a re-circulation system, the process, comprising:
(a) suspending the first polymorph in a solution to form a slurry in the vessel,
(b) re-circulating the slurry, and
(c) removing the slurry from the vessel.

2. The process of claim 1 wherein said second polymorph is thermodynamically more stable.

3. The process of claim 1 wherein said first polymorph is a solvate form and said second polymorph is an anhydrous form.

4. The process of claim 1 wherein the particle size D[90] of said first polymorph is greater than about 60 μm, and the particle size D[90] of said second polymorph is less than about 50 μm.

5. The process of claim 1 wherein said first polymorph consists of large crystals with a particle size D[90] greater than about 150 μm, and said second polymorph consists of small crystals with a particle size D[90] less than about 30 μm.

6. The process of claim 1 wherein said chemical material is 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxamide.

7. The process of claim 6 wherein said first polymorph is a dihydrate form and said second polymorph is an anhydrous form.

8. The process of claim 1 wherein the slurry is re-circulated through a homogenization apparatus in which a high-shear mixing force is applied.

9. The process of claim 8 wherein the homogenization apparatus comprises a stator and a rotatable rotor, and the high-shear mixing force is applied by rotating the rotor at a speed of more than 250 rpm.

10. The process of claim 9 wherein the high-shear mixing force is applied by rotating the rotor at a speed of more than 500 rpm.

11. The process of claim 9 wherein the high-shear mixing force is applied by rotating the rotor at a speed of more than 1,000 rpm.

12. The process of claim 11 wherein said chemical material is 1-(4-methoxyphenyl)-7-oxo-6-(4-(2-oxopiperidin-1-yl)phenyl)-4,5,6,7-tetrahydro-1H-pyrazolo[3,4-c]pyridine-3-carboxamide.

13. The process of claim 12, wherein said first polymorph is a dihydrate form and said second polymorph is an anhydrous form.

14. A process for transforming a first polymorph of a chemical material into a second polymorph of the same chemical material, utilizing an apparatus comprising a vessel connected to a re-circulation system, the process comprising the steps of:
(a) mixing a first solution into a second solution in the vessel to form a slurry of the first polymorph, wherein the first solution comprises the chemical material dissolved in a solvent and the second solution comprises an anti-solvent;
(b) re-circulating the slurry, and
(c) removing the slurry from the vessel.

15. A process for transforming a first polymorph of a chemical material into a second polymorph of the same chemical material, utilizing an apparatus comprising a first vessel, a second vessel, and a third vessel, the process comprising the steps of:
(a) mixing a first solution into a second solution in the first vessel to form a slurry of the first polymorph, wherein the first solution comprises the chemical material dissolved in a solvent and the second solution comprises an anti-solvent;
(b) transferring the slurry into the second vessel wherein a high-shear mixing force is applied on the slurry via a homogenizer apparatus;
(c) transferring the resulting slurry from the second vessel into the third vessel and agitating the slurry; and,
(d) removing the slurry from the third vessel.

16. The process of claim 15, wherein the first vessel is connected to a second vessel via a conduit and a pump, and the second vessel is further connected to a third vessel via a conduit and a pump.

17. The process of claim 15, wherein steps (a)-(d) are carried out continuously.

18. An apparatus for transforming a first polymorph of a chemical material into a second polymorph of the same chemical material, comprising: a vessel connected to a re-circulation system; a means for suspending the first polymorph in a solution to form a slurry in the vessel; and a means for re-circulating the slurry.

19. The apparatus of claim 18, wherein said re-circulation system, comprises: a homogenization apparatus; an outlet means for transferring the slurry from the vessel to the homogenization apparatus; and an inlet means for receiving the slurry from the homogenization apparatus into the vessel.

20. The apparatus of claim 19, wherein said homogenization apparatus comprises a stator and a rotatable rotor, and means for applying a high-shear mixing force by rotating the rotor.

21. An apparatus for transforming a first polymorph of a chemical material into a second polymorph of the same chemical material, comprising: a first source of a first solution; a second source of a second solution; a vessel connected to a re-circulation system; a means for mixing the first solution with the second solution in the vessel to form a slurry of the first polymorph in the vessel; and a means for re-circulating the slurry.

22. The apparatus of claim 21, wherein the first solution comprises the chemical material dissolved in a solvent and the second solution comprises an anti-solvent

23. An apparatus for transforming a first polymorph of a chemical material into a second polymorph of the same chemical material, comprising: a first source of a first solution; a second source of a second solution; a first vessel; a second vessel; a third vessel; a means for mixing the first solution with the second solution in the first vessel to form a slurry of the first polymorph in the first vessel; a means for transferring the slurry in the first vessel into the second vessel; a means for applying a high-shear mixing force on the slurry in the second vessel via a homogenizer apparatus; a means for transferring the resulting slurry from the second vessel into the third vessel, and a means for applying agitation on the slurry in the third vessel.
